SEQUENCE LISTING (4 sequences)

SEQ ID NO: 1 - DNA, 27 bp, Artificial Sequence
  Synthetic construct - DNA probe
tgtaatgatgagggtgtcactgcggtt

SEQ ID NO: 2 - RNA, 27 nt, Artificial Sequence
  Synthetic construct - RNA probe
uguaaugaugagggugucacugcgguu

SEQ ID NO: 3 - DNA, 27 bp, Artificial Sequence
  Synthetic construct - complementary DNA target
aaccgcagtgacaccctcatcattaca

SEQ ID NO: 4 - RNA, 27 nt, Artificial Sequence
  Synthetic construct - complementary RNA target
aaccgcagugacacccucaucauuaca

(19) United States
(12) Patent Application Publication
(10) Pub. No.: US 2010/0086976 A1
(43) Pub. Date: Apr. 8, 2010

(54) METHOD FOR DECONTAMINATING A SOLUTION WITH RESPECT TO UNWANTED NUCLEIC ACIDS

(75) Inventors: Glaucia Paranhos-Baccala, Lyon (FR); Alain Laurent, Grenoble (FR); Ali Laayoun, Colombe (FR)

Correspondence Address: OLIFF & BERRIDGE, PLC, P.O. BOX 320850, ALEXANDRIA, VA, 22320-4850, US

(73) Assignee: BioMerieux, Marcy L'Etoile (FR)

(21) Appl. No.: 12/450,020

(22) PCT Filed: Mar. 28, 2008
(86) PCT No.: PCT/FR2008/050540

§ 371 Date: Sep. 8, 2009

(30) Foreign Application Priority Data
Mar. 30, 2007 (FR) FR0754165

(51) Int. Cl. C12P 19/34

(52) U.S. Cl. 435/91.2

(57) ABSTRACT

The present invention concerns a method for decontaminating a solution of any nucleic acid present in said solution, comprising the following steps:
for a sufficient period, subjecting the solution to the action of at least one type of molecule having the property of degrading the nucleic acids by fragmentation, termed a fragmentation molecule, until said nucleic acids, termed contaminating nucleic acids, have been completely degraded; and
stopping the activity of the fragmentation molecule.

The invention also concerns the use of said treated solution.

The preferred application of the invention is in the field of diagnostics.

The present invention relates to a method for decontaminating a solution of undesirable nucleic acids. The present invention also relates to the use of such a treated solution.

Advances in molecular biology have made it possible to manipulate nucleic acids. Amplification methods have thus become an indispensable tool, meaning that a larger number of nucleic acids can be produced from a very small quantity of nucleic acids in a relatively short time period. A major disadvantage of such techniques resides in the amplification of undesirable nucleic acids, producing erroneous results or even false positives in clinical tests. This is termed contamination.

This contamination may derive from a number of sources: poor cleaning of laboratory benches, personnel, the environment, equipment and pipetting devices, or non-sterile samples.

A certain number of recommendations aimed at limiting this contamination have been developed. They are preventative methods concerning, for example, the handling of samples (in particular sterilization techniques) or laboratory equipment (physically defined work areas, use of fume hoods, pressure gradient between outside and inside, so that the flow can constantly allow evacuation in the desired direction, etc).

Contamination may also come from amplicons derived from preceding amplifications or from cross-contamination between samples.

Another non-negligible source of contamination resides in the starting material (enzymes, reagents) used in the amplification reactions. Thus, Corless et al discuss the problems caused by contaminated tag polymerase on real time PCR sensitivity for the detection of 16S RNA (J Clin Microbiol, 38(5), 1747-1752 (2000)).

A first way of remedying contamination of the enzymes necessary for amplification consists in enzymatic decontamination methods.

Thus, U.S. Pat. No. 5,418,149 describes a method using uracil-DNA-glycosylase which can degrade the nucleic acids deriving from enzyme-producing cells. That technique exploits the over-expression of a desired protein, in this case a polymerase, in E Coli cells which are deficient in uracil-DNA-glycosylase (UNG) and deoxyuridine triphosphatase (dUTPase). Because of the UNG deficiency, the cell does not eliminate incorporated deoxyuracil bases. Because of the deficiency of dUTPase, the cell increases its deoxyuridine pool. Thus, the culture medium allows the production of the desired protein (polymerase) and the incorporation of deoxyuridine into the nucleic acids. The synthesized proteins are then purified using standard purification techniques and by treatment with uracil-DNA-glycosylase which fragments the uracil residue of the residual nucleic acids. The glycosylase is then inactivated by heat to prevent destruction of the target DNA during an amplification reaction.

That method is specific to the production of recombinant proteins, and so its major disadvantage is due to the use thereof. Further, decontamination is limited to nucleic acids containing uracil. Finally, another possible disadvantage is that the heat treatment induces only partial inactivation of uracil-DNA-glycosylase. Target DNA brought into contact with the protein may thus possibly be destroyed.

Another enzymatic method described by Ashkenas et al (Biotechniques; July 2005; 39(1): 69-73) consists of decontaminating a solution containing all of the elements necessary for amplification. In this case a cocktail of restriction enzymes is used in the context of RT-PCR. Those enzymes degrade the double strand DNA present in the amplification reaction medium containing the target RNA to be amplified. They are then, inactivated by heat when reverse transcription occurs. An alternative to that method for a PCR application, i.e. in the presence of double strand target DNA, is also described, but it is limited to the use of a single type of restriction enzyme (type IIS RE). However, the use of restriction enzymes suffers from the disadvantage that it only cleaves double strand nucleic acids which in addition must have a recognition site. For this reason, contaminants in the form of a single strand and/or having few or no such sites are not eliminated thereby.

Other enzymatic decontamination methods consist of eliminating undesirable nucleic acids from a solution before being brought into contact with target nucleic acids. Patent application WO-A-99/07887 describes the use of a thermolabile DNAse which can degrade the double strand nucleic acids contained in the reaction medium before contact with the target nucleic acids. The enzyme is then inactivated by heat.

The major disadvantage with that technique is that this enzyme cannot degrade contaminants in the form of RNA, single strand DNA or RNA/DNA heteroduplexes.

Further, inactivation of that enzyme by heat in the reaction medium necessitates the use of thermostable polymerases.

The prior art also deals with non-enzymatic methods.

Thus, Mohammadi et al (J Clin Microbiol, October 2003; 41(10): 4796-8) describes a technique consisting of column filtration of extraction reagents and optional digestion by a restriction enzyme, Sau3AI, of the PCR reagents before amplification. The disadvantage with filtration techniques resides in the fact that such techniques cannot be applied to complex media without changing either the concentration or the properties. Further, such a supplemental filtration step may itself be a source of contamination.

Patent application WO-A-94/12515 describes a method for treatment by a photoreactive compound of a solution containing Taq polymerase and potentially contaminating nucleic acids. That photoreactive compound, for example a furocoumarin derivative, is activated by exposure to ultraviolet radiation. A major disadvantage with that technique, apart from being difficult to use, is the deleterious effect of radiation on proteins. Further, that method remains of low effectiveness because of the random degradation of said nucleic acids, possibly generating fragments which may also be amplified.

Another non-enzymatic method described by Chang et al (RNA, May 2005; 11(5): 831-6) uses a cobalt complex to inhibit translation. That complex can hydrolyze phosphate diester bonds of DNA and RNA.

The major disadvantages with that technique are the incomplete degradation of the nucleic acids and the slow decontamination reaction (24 hours).

Metal complexes have also been described in patent application WO-A-2006/034009 in order to purify biological molecules contaminated by nucleic acids. Thus, a fragmentation complex constituted by two phenanthroline molecules associated with a copper atom (bis(1,10-phenanthroline/Cu) was used to purify a solution containing tag polymerase. That solution was then purified using standard chromatographic techniques in order to remove the contaminating nucleic acids and/or fragmentation molecules.

The major disadvantage with that method is the presence of a purification step rendering its use difficult and increasing the risk of fresh contamination.

Patent EP-B-0 646 180 describes a method for inactivating nucleotide sequences using metal chelates. A fragmentation complex such as bis(1,10-phenanthroline)/Cu was used to decontaminate the solutions, after the amplification step, of all of the nucleic acids present. This could prevent contamination of new samples by amplicons derived from a previous amplification reaction. That document also describes the use of a fragmentation molecule to decontaminate biological product preparations.

In the case of the first use, a major disadvantage of that method is that it cannot degrade all of the contaminating nucleic acids but only amplicons derived from a previous amplification reaction. In the second case, the major disadvantage is the presence of a purification step (ultrafiltration, precipitation, chromatography), rendering its use difficult and increasing the risk of fresh contamination.

Having regard to the disadvantages of the prior art, the inventors propose a novel method which is simple and rapid for decontaminating a solution of undesirable nucleic acids.

In this context, in a first aspect the present invention provides a method for decontaminating a solution of any nucleic acid present in said solution, comprising the following steps:
for a sufficient period, subjecting the solution to the action of at least one type of molecule having the property of degrading the nucleic acids by fragmentation, termed a fragmentation molecule, until said nucleic acids, termed contaminating nucleic acids, have been completely degraded; and
stopping the activity of the fragmentation molecule.

One of the advantages of the present invention is that this decontamination method does not necessitate a step for purification to eliminate the fragmentation molecules.

The nucleic acids which are treated are RNA or DNA, in the single or double strand form, as well as RNA/DNA heteroduplexes.

This method is particularly useful for decontaminating starting materials such as, for example, the enzymes from natural sources such as bacteria. These enzymes are thus highly contaminated with natural nucleic acids derived from said bacteria.

In another aspect, the present invention provides a method for decontaminating a solution containing contaminating nucleic acids and nucleic acids of interest, and comprising the steps defined above, namely:
for a sufficient period, subjecting the solution to the action of at least one type of molecule having the property of degrading the contaminating nucleic acids by fragmentation, termed a fragmentation molecule, until said contaminating nucleic acids have been degraded, while conserving the nucleic acids of interest;
stopping the activity of the fragmentation molecule.

The contaminating nucleic acids may be human genomic DNA and the nucleic acids of interest may be viral or bacterial nucleic acids present in the mixture in very small quantities. Said nucleic acids of interest may be protected from the action of the fragmentation molecule. As an example, they may be naturally protected by an outer envelope (capsid, bacterial wall, etc), while the contaminating nucleic acids are not.

In one advantageous implementation of the invention, after stopping the activity of the fragmentation molecule, the method comprises a supplemental step which consists in an amplification reaction.

Preferably, the decontamination method of the invention is characterized in that the fragmentation molecule used has the property of hydrolyzing the phosphate diester bonds of the contaminating nucleic acids in a random manner.

In accordance with one implementation, the fragmentation molecule is a complex constituted by two molecules of 1,10-phenanthroline associated with a metal atom forming the bis-(1,10-phenanthroline)/metal complex. Preferably, the metal is a transition metal such as copper, ruthenium, nickel, iron, zinc, rhodium, cobalt or manganese.

Advantageously, the two phenanthroline nuclei of the fragmentation molecule are connected to each other via a linking arm to form a ClipPhen molecule.

ClipPhen molecules have been widely described in the literature. Various linking arms connect the two phenanthroline nuclei. Advantageously, the linking arm between the two phenanthroline nuclei is constituted by a chain of three successive carbon atoms wherein the carbon atom in the central position is substituted and wherein each terminal carbon atom is connected to a phenanthroline nucleus via an oxygen atom, such as serinol. Preferably, the carbon atom in the central position is substituted with NH₂ or NH—CO—CH₃, and each terminal carbon atom is connected to a phenanthroline nucleus via an oxygen atom in position 2 or 3 of said nucleus.

Many other linkages are possible such as, for example, diamino-cyclohexane (Chemistry Letters, vol 33, n^{o} 6, p 684-985, Hayashi et al) or an ethanediol, a propanediol or a pentanediol (Synlett 2001, n^{o} 10, 1629-1631, Boldron et al).

The fragmentation activity of the ClipPhen molecule associated with a metal is also known to the skilled person (Chemical Review, 1993, 93, 2295-2316, D Sigman et al; J Chem Soc 1961, 2007-2019, James et al). This molecule has been examined in a number of studies with the aim of improving its hydrolysis properties. It has the advantage of degrading all nucleic acids, i.e. DNA and RNA, in the single or double strand form as well as RNA/DNA heteroduplexes. Thus, 2-ClipPhen/metal ((1,3-bis(1,10-phenanthrolin-2-yloxy)propan-2-amine)/metal) and 3-ClipPhen/metal ((1,3-bis(1,10-phenanthrolin-3-yloxy)propan-2-amine)/metal) have exhibited nuclease activity which is greatly augmented compared with the bis(1,10-phenanthroline)/metal complex (Inorganic Chemistry 1998, 3486-3489, Pitié et al; Chem Commun 1998, 2597-2598, Pitié et al; Eur J Inorg Chem 2003, 528-540, Pitié et al and Bioconjugate Chemistry 2000, 11, 892-900, Pitié M et al). Similarly, a ClipPhen molecule complexed with copper can hydrolyze very effectively and in a random manner the phosphate diester bonds of DNA or RNA. It can complex with a copper atom to produce complexes with an oxidation state of I or II as shown in the publication by James et al, J Chem Soc 1961, 2007-2019. The molecules 2-ClipPhen-Cu and 3-ClipPhen-Cu are shown below:

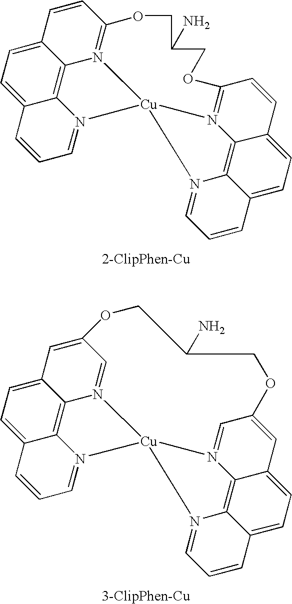

Thus, the present invention also pertains to a method for decontaminating a solution of any nucleic acid present in said solution, consisting of subjecting the solution to the action of the ClipPhen/metal molecule for a sufficient period until the contaminating nucleic acids have been completely degraded.

In a further aspect, the present invention consists in a method for decontaminating a solution containing contaminating nucleic acids and nucleic acids of interest, consisting of subjecting the solution to the action of the ClipPhen/metal molecule for a sufficient period until said contaminating nucleic acids have been degraded, while conserving the nucleic acids of interest.

In accordance with one implementation of the present invention, the fragmentation activity of the fragmentation molecule is stopped by adding an excess of organic reducing agent. When the organic reducing agent is constituted by a thiol, the ratio between the fragmentation molecule and the organic reducing agent stopping the reaction is more than 1 to 100 and preferably more than 1 to 1000.

One advantage of this implementation is that the fragmentation molecules are inactivated in situ. These molecules thus do not need to be removed from the solution. This latter could be used as is in various applications, such as amplification, for example.

Thus, depending on the concentration of the organic reducing agent present, the fragmentation molecules may be activated or deactivated.

In accordance with another implementation of the present invention, the fragmentation activity of the fragmentation molecule is stopped by adding a complexing agent (CA) such as EDTA, for example, or any other complexing agent with more affinity for the metal than ClipPhen and which can displace the equilibrium:

ClipPhen-metal+CA→ClipPhen+metal-CA

In one particular implementation of the present invention, the fragmentation molecule is a type I copper complex associated with hydrogen peroxide, H₂O₂, and optionally with another reducing agent, preferably organic. Preferably, the fragmentation molecule is a ClipPhen/Cu I molecule.

In the case of a type I copper complex, the nuclease activity proceeds in two stages:
a) the positively charged copper complex forms a complex with the nucleic acid;
b) the nucleic acid is oxidized by formation of a Cu-oxo or Cu—OH intermediate with H₂O₂ which, after a series of reactions, results in cleavage of the nucleic acid.

In accordance with another implementation, the molecule is a type II copper complex associated with another reducing agent, preferably organic. In this case, the hydrogen peroxide is generated in situ under the action of air and the reducing agent. Preferably again, the fragmentation molecule is a ClipPhen/Cu II molecule.

When the other organic reducing agent is a carboxylic acid or a derivative of that acid, the ratio between the fragmentation molecule and the organic reducing agent is in the range between 1 to 1 and 1 to 100.

As a function of the preceding implementations, the organic reducing agent is constituted by:
a thiol such as dithiothreitol (DTT), a thio-acid, such as mercaptopropionic acid; or
a carboxylic acid; or
a derivative of a carboxylic acid such as the ascorbate; or
a phosphine such as tricarboxyethyl phosphine (TCEP).

In a particular implementation, the fragmentation molecule and/or the reducing agent is immobilized on a solid support. This solid support may be a particle of latex (optionally magnetic), glass (CPG), silica, polystyrene, agarose, sepharose, nylon, etc. It may also be a filter, a membrane, a strip or a film.

The fragmentation activity of the fragmentation molecule may also be stopped when the solid support on which the fragmentation molecule and/or the reducing agent is fixed is removed from the solution.

The fragmentation molecule may be fixed by covalent bonding or by adsorption onto the supports under consideration. Many references concerning supported reagents exist; reference may in particular be made to Laurent et al, Tetrahedron Letters 45, 8883-8887, 2004.

Another advantage of the present invention over the prior art is the rapidity of the reaction undergone by the nucleic acids. The treatment period is in the range 5 minutes to 60 minutes, preferably in the range 10 minutes to 30 minutes, for concentrations of ClipPhen/metal molecules in the range 1 nM to 100 μM.

In a preferred mode of the invention, the solution contains all of the constituents necessary for an amplification reaction with the exception of the nucleic acids, i.e. the targets, the amplification primers and the detection probes.

The present invention also pertains to the use of a solution treated in accordance with the present invention, the solution being mixed with a biological sample containing target nucleic acids which are to be amplified, but also amplification primers and detection probes which are specific for the target nucleic acids in the presence of the organic reducing agent, thereby creating an excess of organic reducing agent which stops the action of the fragmentation molecules.

The organic reducing agent which stops the action of the fragmentation molecules may be identical to or different from the other organic reducing agent which has been added to activate the nuclease reaction of the fragmentation molecule.

### DEFINITIONS

The term “nucleic acid” means a concatenation of at least two deoxyribonucleotides or ribonucleotides.

The nucleic acid may be natural or synthetic, an oligonucleotide, a polynucleotide, a nucleic acid fragment, a genomic DNA, a ribosomic RNA, a messenger RNA, a transfer RNA, or a nucleic acid obtained by an enzymatic amplification technique, such as:
PCR (Polymerase Chain Reaction), described in U.S. Pat. No. 4,683,195, U.S. Pat. No. 4,683,202 and U.S. Pat. No. 4,800,159, and its derivative RT-PCR (Reverse Transcription PCR), in particular as described in a single-step format, such as that described in patent EP-B-0 569 272;
LCR (Ligase Chain Reaction), described, for example, in patent application EP-A-0 201 184;
RCR (Repair Chain Reaction), described in patent application WO-A-90/01069;
3SR (Self Sustained Sequence Replication), as in patent application WO-A-90/06995;
NASBA (Nucleic Acid Sequence-Based Amplification), as in patent application WO-A-91/02818; and
TMA (Transcription Mediated Amplification), as described in U.S. Pat. No. 5,399,491.

We shall use the term “amplicons” to designate the nucleic acids generated by an enzymatic amplification technique.

The term “any nucleic acid” means sequences of RNA or DNA, in the single or double strand form, as well as RNA/DNA heteroduplexes.

The term “stop the activity of the fragmentation molecule” means:
adding an excess of organic reducing agent; or
adding a complexing agent.

The term “degradation”., means the degradation or fragmentation of at least 90% of the contaminating nucleic acids. Preferably, the degradation is termed “complete”, which means that the degradation of DNA or RNA, preferably by hydrolysis of phosphate diester bonds, causes the formation of “non-amplifiable” nucleic acid sequences.

The term “non-amplifiable” nucleic acids means nucleic acids which cannot be amplified in the presence of primers with a preferred size of at least 10 nucleotides. The size of these “non-amplifiable” nucleic acids is thus less than 20 nucleotides, preferably less than 10 nucleotides.

The term “solution” means a homogeneous or heterogeneous aqueous solution. It may be a complex solution containing enzymes, organic molecules (triphosphate nucleosides, sugars, saccharides, polysaccharides, small organic molecules, etc), inorganic molecules (salts, etc), as a mixture or otherwise. The solution may also contain a solid support such as particles which may be formed from latex, optionally magnetic, glass (CPG), silica, polystyrene, agarose, sepharose, nylon, etc. It may also be a filter, a film, a membrane or a strip.

The solution may also be constituted by a solution in contact with a solid surface such as a plastic tube (Eppendorf type), thereby allowing decontamination of said surface.

The accompanying Figures and examples show particular implementations and should not be considered to limit the scope of the present invention.

FIG. 1: Bioanalyzer study (Agilent, RNA 6000 Pico Kit, USA) of the degradation of a 1080 base transcript (wild type Salmonella) subjected to the action of ClipPhen-Cu/ascorbate mixture for 1 hour at 37° C. The dark band located at 31 seconds in columns 2, 3, 4, 6 and 7 corresponds to the 1080 base transcript.
Column 1: nucleotide size scale. Injection of a series of increasing RNA sizes, allowing quantification and computation of the size of the analyzed nucleic acids, as a function of the time at which they pass in front of the bioanalyzer detector. The unit of measurement on the vertical axis is a second. The conversion between the passage time and the mass of the nucleic acids is carried out by the bioanalyzer software.
Column 2: 5 nM (nanomolar) transcript in the presence of 5 μM (micromolar) ClipPhen-Cu.
Column 3: 5 nM transcript in the presence of 500 nM ClipPhen-Cu.
Column 4: 5 nM transcript in the presence of 50 nM ClipPhen-Cu.
Column 5: idem column 2 with addition of 500 μM ascorbate.
Column 6: idem column 3 with addition of 50 μM ascorbate.
Column 7: idem column 4 with addition of 5 μM ascorbate.

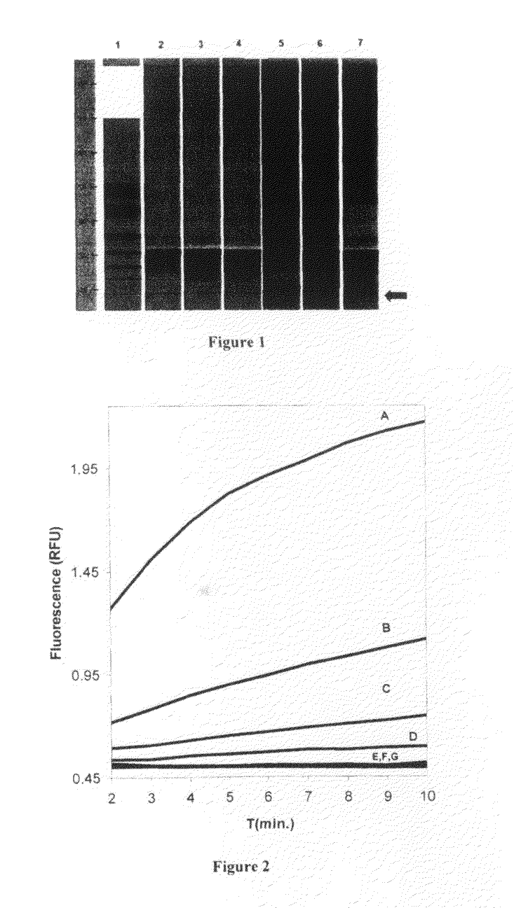

FIG. 2: Inhibition of fragmentation of nucleic acids by increasing the concentration of DTT (0 to 16 mM).

Curve A corresponds to 0 mM of DTT.

Curve B corresponds to 1 mM of DTT.

Curve C corresponds to 3 mM of DTT.

Curve D corresponds to 5 mM of DTT.

Curve E corresponds to 10 mM of DTT.

Curve F corresponds to 16 Mk of DTT.

Curve G corresponds to the control: 0 mM of DTT and 0 mM of ascorbate.

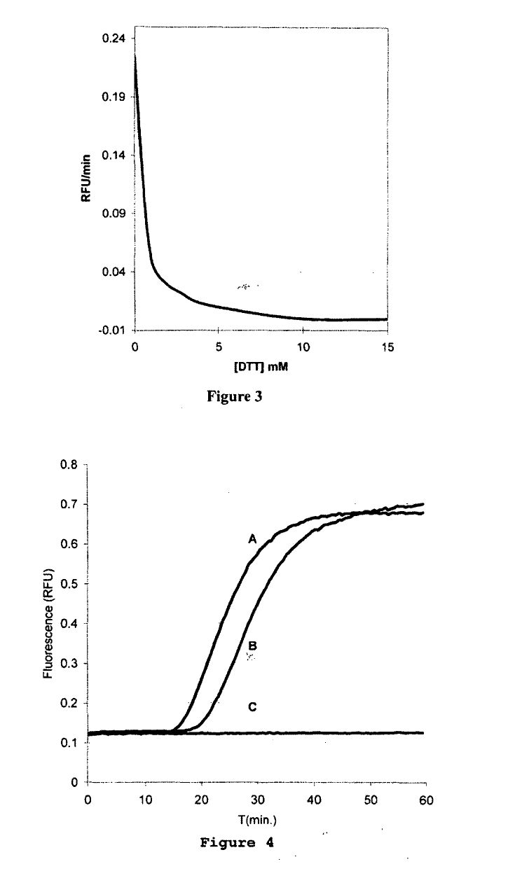

FIG. 3: Rate of fragmentation of model fluorogenic oligonucleotide as a function of concentration of DTT in the mixture (in relative fluorescence units (RFU) per minute).

FIG. 4: Effect of ClipPhen-Cu/ascorbate mixture on an aqueous solution containing nucleic acids (100 cell equivalent/μl total RNA of B Cereus).

Curve A corresponds to the amplification control which has not undergone any treatment (+control).

Curve B corresponds to the 50 μM ClipPhen-Cu/500 μM ascorbate mixture the activity of which was immediately inhibited by adding 17 mM of DTT and which was incubated for 30 minutes.

Curve C corresponds to the 50 μM ClipPhen-Cu/500 μM ascorbate mixture incubated for 30 minutes, stopping fragmentation with DTT after this time.

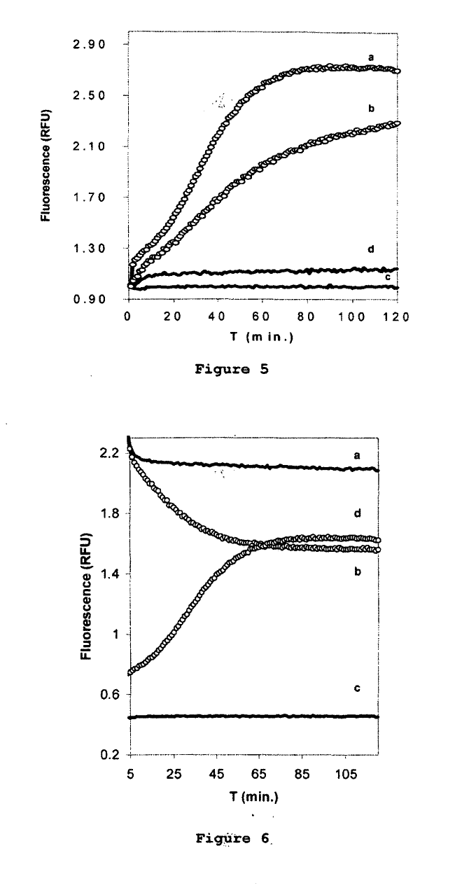

FIG. 5: Comparison of fragmentation of DNA or single strand RNA by ClipPhen-Cu (with or without ascorbate). Curves normalized to 1.

Curve a: Fluorogenic oligonucleotide (DNA) n^{o} 16025 subjected to the action of ClipPhen-Cu/ascorbate mixture.

Curve b: Fluorogenic oligonucleotide (RNA) n^{o} 16027 subjected to the action of ClipPhen-Cu/ascorbate mixture.

Curve c: Negative control for experiment a: fluorogenic oligonucleotide (DNA) n^{o} 16025 subjected to the action of ClipPhen-Cu mixture alone.

Curve d: Negative control for experiment b: fluorogenic oligonucleotide (RNA) n^{o} 16027 subjected to the action of ClipPhen-Cu mixture alone.

FIG. 6: Comparison of single or double strand DNA fragmentation by the ClipPhen-Cu mixture (with or without ascorbate). The curves represent a fragmentation rate corresponding to an increase in the fluorescence per minute (RFU/min).

Curve a: Fluorogenic'oligonucleotide (DNA) n^{o} 16025/complementary DNA target (n^{o} 16026) duplex subjected to the action of ClipPhen-Cu mixture (negative control).

Curve b: Fluorogenic oligonucleotide (DNA) n^{o} 16025/complementary DNA target (n^{o} 16026) duplex subjected to the action of ClipPhen-Cu/ascorbate mixture.

Curve c: Fluorogenic oligonucleotide (DNA) n^{o} 16025 subjected to the action of ClipPhen-Cu mixture (negative control).

Curve d: Fluorogenic oligonucleotide (DNA) n^{o} 16025 subjected to the action of ClipPhen-Cu/ascorbate mixture.

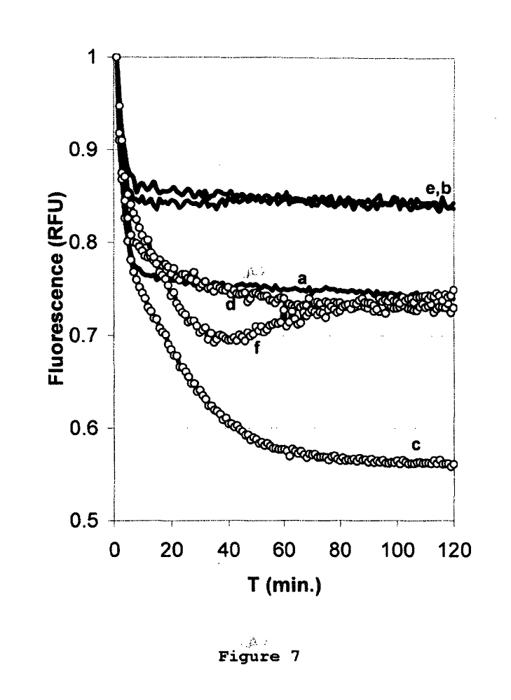

FIG. 7: Comparison of fragmentation of DNA or RNA duplex or a RNA/DNA heteroduplex by ClipPhen-Cu (with or without ascorbate). Curves normalized to 1.

Curve a: Fluorogenic oligonucleotide (DNA) n^{o} 16025/complementary DNA target (n^{o} 16026) duplex subjected to the action of ClipPhen-Cu mixture (negative control).

Curve b: Fluorogenic oligonucleotide (RNA) n^{o} 16027/complementary RNA target (n^{o} 16028) duplex subjected to the action of ClipPhen-Cu mixture (negative control).

Curve c: Fluorogenic oligonucleotide (DNA) n^{o} 16025/complementary DNA target (n^{o} 16026) duplex subjected to the action of ClipPhen-Cu/ascorbate mixture.

Curve d: Fluorogenic oligonucleotide (RNA) n^{o} 16027/complementary RNA target (n^{o} 16028) duplex subjected to the action of ClipPhen-Cu/ascorbate mixture.

Curve e: Fluorogenic oligonucleotide (RNA) n^{o} 16027/complementary DNA target (n^{o} 16026) heteroduplex subjected to the action of ClipPhen-Cu mixture (negative control).

Curve f: Fluorogenic oligonucleotide (RNA) n^{o} 16027/complementary DNA target (n^{o} 16026) heteroduplex subjected to the action of ClipPhen-Cu/ascorbate mixture.

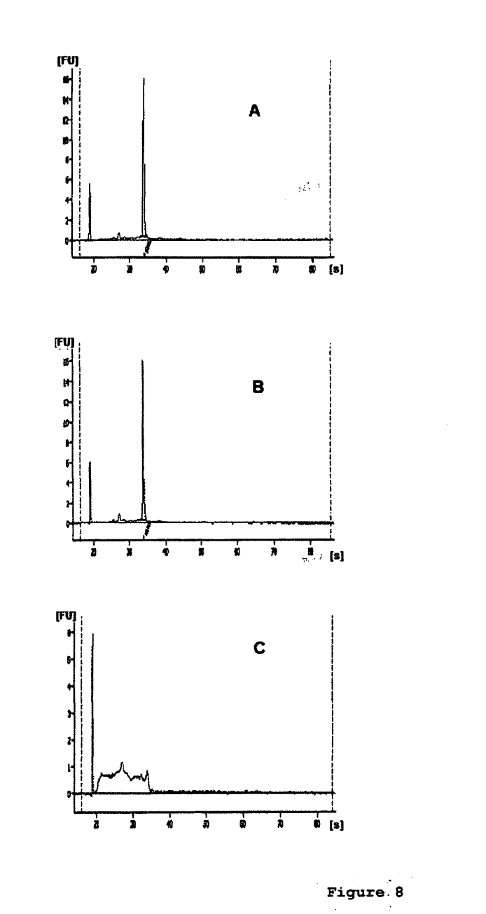

FIG. 8: Bioanalyzer (Agilent) observation of cleavage of 16s RNA (13 ng/μL) by ClipPhen-Cu immobilized on a Tentagel bead in the presence of 5 mM ascorbate.

Electrophoregram A: RNA target before degradation in water.

Electrophoregram B: RNA target before degradation in the presence of ascorbate.

Electrophoregram C: RNA target degraded in the presence of ascorbate and ClipPhen-Cu immobilized on Tentagel resin (1 bead of Tentagel, 30 min).

### EXAMPLE 1

### Hydrolysis of a Transcript by the ClipPhen-Cu Molecule, and Initiator Effect of Ascorbate

Aim: This example demonstrates the fragmentation action of a ClipPhen-Cu/ascorbate mixture on a model nucleic acid (Salmonella RNA transcript: 1080 bases). It also demonstrates the effect of ascorbate on the initiation of the fragmentation reaction. The transcript was subjected to the action of various concentrations of ClipPhen-Cu/ascorbate mixture. The control was also subjected to the action of ClipPhen-Cu but in the absence of ascorbate.

The fragments were then analyzed using a Bioanalyzer from Agilent (FIG. 1).

### Operating Procedure:

A solution of transcript (Salmonella wild type, 1080 bases) was constituted from 2 μl of transcript in a concentration of 1 μM in water and 18 μl of 80 mM (millimolar) phosphate buffer at a pH of 7.2, MgCl₂ 20 mM, NaCl 100 mM. This solution was heated to 70° C. for 2 minutes then cast into ice.

Next, 5 μl of this solution to which 1 μl of ClipPhen-Cu (500, 50 and 5 μM) and 4 μl of the buffer used above were added. Solution 2 was obtained which was left for 1 h at 37° C.

Next, 5 μl of solution 2 was taken and 42.5 μl of buffer and 2.5 μl of a freshly prepared ascorbate solution (100 eq with respect to the [ClipPhen-Cu]) or water in the case of the controls were added.

Thus, a series of tubes was prepared respectively comprising 5 nM of transcript, (5 μM, 500 nM and 50 nM) of ClipPhen-Cu and (500, 50 and 5 μM) of ascorbate or water for the controls.

Immediately, 1 μl of the 6 solutions was injected over the RNA 6000 Pico chip (commercial kit with reference number 5067-1512, Agilent, USA, used on the BioAnalyzer system from Agilent, USA) in order to observe the fragmentation products.

### Results and Conclusions:

The effect of fragmentation on the transcript, columns 5 to 7, was observed by the appearance of bands with various sizes below 20 nucleotides (arrow on right hand side at 20 seconds). Thus, the transcript which had undergone the treatment with the ClipPhen-Cu/ascorbate mixture was partially or completely degraded as a function of the concentration of ClipPhen-Cu. Between 5 μM and 0.5 μM, the initial band was observed to disappear. The reaction time for obtaining complete degradation of all of the amplicons was of the order of 30-60 minutes with 5 nM of transcript, 5 μM of ClipPhen-Cu and 500 μM of ascorbate. The quantity of ClipPhen-Cu for total degradation of the transcript was of the order of micromolar (column 5).

In contrast, the same experiment carried out without ascorbate (column 2) clearly showed an intact band.

This thus demonstrates that the ClipPhen-Cu mixture has no activity when ascorbate has not been added (excess of the order of 1-100 equivalents).

### EXAMPLE 2

### Inhibition of ClipPhen-Cu Molecule by a Reducing Agent

### Aim:

This example is intended to show that degradation of nucleic acids by the ClipPhen-Cu/ascorbate mixture can be completely inhibited by adding an excess of a reducing agent such as DTT.

A model fluorogenic oligonucleotide (Seq 16025 synthesized by Eurogentec (Liège, Belgium): 5′ (6-FAM) TGT AAT GAT GAG GGT GTC ACT GCG GTT (TAMRA)) was subjected to the action of a ClipPhen-Cu/ascorbate mixture. By fragmenting, it allowed a fluorescence to appear at 520 nm since the FAM fluorophore was physically at a distance from the TAMRA fluorescence extinguisher. The appearance of fluorescence was measured with time using a fluorimeter (NucliSens EasyQ Analyzer, Nasba Diagnostic, BioMerieux, Boxtel (NL)).

The experiment was carried out with increasing concentrations of DTT for a fixed concentration of ClipPhen-Cu/ascorbate in order to measure the inhibition provided by DTT by attenuation of the emitted fluorescence.

### Operating Procedure:

A series of tubes containing the elements below was incubated at 37° C. for 5 minutes:
the fluorogenic oligonucleotide 16025, 10 nM (1 μl to 200 nM);
DTT (1 μl at a concentration to produce a series from 1 to 16 mM in the final volume); and
17 μl of phosphate buffer, 80 mM, pH 7.2, MgCl₂ 20 mM, NaCl 100 mM.

Next, 1 μl of a ClipPhen-Cu/ascorbate mixture prepared as follows was added: 200 μM of ClipPhen and 200 μM of CuCl₂ in 50/50 DMF/water and 20 mM ascorbate in water, prepared extemporaneously (from concentrated solutions of 10 mM of ClipPhen-Cu and 1 M of ascorbate).

Finally, the 20 μl of solution contained: 10 nM of fluorogenic oligonucleotide, 10 μM of ClipPhen+Cu²⁺, 1 mM of ascorbate and 0/1/3/5/10/16 mM of DTT.

A control was also produced without ascorbate and without ClipPhen-Cu. Next, the emitted fluorescence was measured over 60 minutes (FIG. 2). The conditions were as follows: exc/em filters (485/518 nm), time interval (1 min), integration time (100 ms).

Using the curves obtained, the fragmentation reaction rate over the first two minutes of the reaction was measured and the graph, FIG. 3, representing the fragmentation rate as a function of the DTT concentration was traced.

### Results and Conclusions:

It can be seen that for increasing concentrations of DTT, fluorescence no longer appeared from 10 mM (FIGS. 2 and 3). This means that the activity of the ClipPhen-Cu/ascorbate mixture is completely stopped by excess reducing agent. This experiment demonstrates that adding 10-16 mM of DTT to the nucleic acid/ClipPhen-Cu/ascorbate mixture can stop the reaction and render this mixture compatible with a future use in an amplification reaction.

### EXAMPLE 3

### Decontaminating Effect of ClipPhen in an Aqueous Solution

### Aim:

Decontamination of an aqueous solution contaminated by a total RNA mixture from B Cereus in an amount of 200 cell equivalent/μl subjected to the action of a ClipPhen-Cu/ascorbate mixture (50 μM/500 μM) for 30 minutes. The activity of the ClipPhen-Cu was then completely stopped by adding DTT in an amount of 10 mM. Next, a NASBA amplification reaction (Nuclisens Basic Kit V2 from bioMérieux, reference 60079136, Boxtel (NL)) was carried out in order to evaluate the fragmentation efficiency and demonstrate the absence of amplifiable nucleic acids. It was not necessary to carry out purification prior to amplification on this mixture. A control was produced in which the activity of the ClipPhen-Cu was inhibited from t0 in order to measure any inhibiting effect of this latter. To this end, DTT was added under sufficient conditions (10 mM) before adding the target RNA and kept under incubation for 30 minutes.

### Operating Procedure:

### Decontamination by the ClipPhen-Cu/Ascorbate Mixture of an Aqueous Solution Enriched in Total RNA

10 μl of ClipPhen-Cu (250 μM in water/DMF, 50/50), 20 μl of water and 10 μl of ascorbate in a concentration of 2.5 mM in water were added to a total RNA solution of RNA from B Cereus (5 μl at 2000 cell equivalent/μl in water).

This solution to be decontaminated was incubated for 30 minutes at ambient temperature.

### Stopping Fragmentation Activity of ClipPhen-Cu/Ascorbate Mixture

5 μl of 173.7 mM DTT (final DTT concentration: 17.3 mM) was added to the preceding solution to stop the hydrolysis reaction and deactivate the ClipPhen. Solution A was obtained.

Control to Show that the ClipPhen-Cu/Ascorbate/DTT Mixture has No Fragmentation Activity and does not Inhibit NASBA if the Fragmentation Activity is Stopped Immediately by Adding DTT

At the same time, a control experiment was carried out in which a total RNA solution of RNA from B Cereus (5 μl at 2000 cell equivalent/μl in water) was incubated in the presence of 5 μl of DTT at 173.7 mM, 10 μl of ClipPhen-Cu (250 μM in water/DMF 50/50, 20 μl of water and 10 μl of ascorbate, 2.5 mM in water). This solution, in which the ClipPhen-Cu/ascorbate mixture was immediately inactivated, was incubated for 30 minutes at ambient temperature.

This experiment could measure any inhibition of the amplification reaction by the ClipPhen-Cu/ascorbate/DTT mixture. This was solution B.

Amplification of Nucleic Acids Remaining in Solution A after Decontamination and Still Present in Solution B

Next, 5 μl of solutions A or B to which 10 μl of the “Reactive Mix: REAG” (from NucliSens EasyQ Basic Kit bioMérieux, trade reference 285006, Boxtel (NL)) was taken. This mixture contained the reagents necessary for amplification, two primers and the probe necessary for detection of amplification. This solution also contained sufficient DTT (6 mM) to prevent reactivation of the ClipPhen. The final concentration of the mixture was thus 10 mM of DTT. The solutions were incubated for 2 minutes at 65° C. then 2 minutes at 41° C.

Next, 5 μl of the “Enzyme Mix: ENZ” mixture from Basic Kit NucliSens bioMérieux was added to the preceding solutions in order to commence amplification. The reaction medium was held at 41° C. for 1 h 30 in the “Nuclisens EasyQ” fluorimeter from bioMérieux (NucliSens EasyQ Analyzer, Nasba Diagnostic, bioMérieux, Boxtel (NL)). The fluorescence measurements were recorded and the curves were traced (FIG. 4).

### Results and Conclusions:

A slight delay was observed with curve B which corresponds to the control which has been subjected to the action of the ClipPhen-Cu/ascorbate mixture inhibited immediately by addition of the DTT solution. This delay in amplification does not change the sensitivity of the test at all since the slopes for amplification initiation are of the same amplitude. It is due to an increase in the concentration of DTT with respect to the positive control (curve A). In this case it was shown that the ClipPhen-Cu/ascorbate/DTT mixture has no effect on the NASBA and that the enzymes for this amplification reaction are not affected by this mixture of compounds. Further, it was also shown that DTT allows inhibition of the fragmentation activity of the ClipPhen-Cu since no degradation activity was noted when sufficient DTT was added.

Curve C shows that while the activity of the ClipPhen-Cu/ascorbate mixture was maintained for 30 minutes then stopped by adding DTT (17 mM), the fragmentation is such that the amplification curve is no longer delayed.

It has been demonstrated here that a complex mixture (ClipPhen-Cu/ascorbate) can be used to degrade nucleic acids, that this activity can be stopped by adding an excess of DTT and this solution (without purification) can be used for subsequent enzymatic amplification. This technique can thus easily overcome problems with contamination by nucleic acids.

### EXAMPLE 4

### Fragmentation by ClipPhen-Cu of Nucleic Acids Other than RNA; Application to Single Strand DNA, Double Strand DNA and Double Strand RNA and to a RNA/DNA Heteroduplex

### Aim:

This example demonstrates that the ClipPhen-Cu/ascorbate mixture can degrade single strand RNA or DNA as well as the corresponding duplexes or heteroduplexes.

### Operating Procedure:

The solutions indicated in the table below were prepared from fluorogenic oligonucleotides (probes) and complementary targets ordered from Eurogentec (Table 1).

TABLE 1
Sequences used in Example 4.

| | | | 5′ | 3′ |
|---|---|---|---|---|
| Notes | ODN Ref | Sequence | modification | modification |
| Fluorogenic | 16025 | TGT AAT GAT GAG GGT GTC ACT GCG GTT | 6-FAM | TAMRA |
| DNA probe | | | | |
| Fluorogenic | 16027 | UGU AAU GAU GAG GGU GUC ACU GCG GUU | 6-FAM | TAMRA |
| RNA probe | | | | |
| Complementary | 16026 | AAC CGC AGT GAC ACC CTC ATC ATT ACA | — | — |
| DNA target | | | | |
| Complementary | 16028 | AAC CGC AGU GAC ACC CUC AUC AUU ACA | — | — |
| RNA target | | | | |

The mother solutions of the fluorogenic oligonucleotides were in a concentration of 200 nM in water which were diluted to 10 nM (1 μl) in 18 μl of 80 mM phosphate buffer, pH 7.2, MgCl₂ 20 mM, NaCl 100 mM. The complementary target was added at the same concentration as a function of the case. The solutions were then denatured and left at 37° C. for one hour to allow the two strands to hybridize. Next, a solution of ClipPhen-Cu (1 μL to 20 μM in water/DMF, [final ClipPhen-Cu]=1 μM) then a solution of ascorbate (1 μl to 2 μM in water [final ascorbate]=100 μM), except for the controls, were then added before commencing the fluorescence measurement using EasyQ (bioMérieux, see Example 1) for 120 minutes at 37° C. These measurements were carried out four times along with the corresponding control experiments containing no ascorbate. Only the mean value is shown in FIGS. 5, 6 and 7.

TABLE 2
Mixtures of oligonucleotides used in Example 4.

| | | Molarity | | | |
|---|---|---|---|---|---|
| | ODN | (mM) in | ClipPhen-Cu | Ascorbate | |
| | mixture | buffer | (μM) | molarity (mM) | |
| Fluorogenic | 16025 | 10 | 1 | 0 | 100 |
| DNA probe | | | | | |
| Fluorogenic | 16027 | 10 | 1 | 0 | 100 |
| RNA probe | | | | | |
| DNA/DNA | 16025/16026 | 10/10 | 1 | 0 | 100 |
| RNA/RNA | 16027/16028 | 10/10 | 1 | 0 | 100 |
| RNA/DNA | 16027/16026 | 10/10 | 1 | 0 | 100 |

### Results and Conclusions:

FIG. 5 shows the results of the fluorescence measurement over time for a single strand fluorogenic DNA (16025, curve a) or fluorogenic RNA (16027, curve b) subjected to the action of a ClipPhen-Cu/ascorbate mixture. In both cases a very clear increase in fluorescence was observed compared with the controls (curves c and d). This appearance of fluorescence demonstrates that both the DNA as well as the RNA were fragmented and at a comparable rate during the first moments of the reaction.

FIG. 6 shows the fragmentation of a DNA/DNA duplex. During formation of the DNA/DNA duplex (curves a and b) we observe that the level of fluorescence is increased by a factor of 6 compared with the single strand control (curves c or d). This increase is not due to ClipPhen-Cu but to hybridization between the two strands. From the moment the ascorbate is added, a reduction in fluorescence in curve b is observed which meets the level of fluorescence of curve d corresponding to the fluorescence emitted by the completely cleaved single strand. Modification of the fluorescence compared with the controls clearly shows that the single strand DNA and double strand DNA/DNA are cleaved in a completely comparable manner.

Finally, we evaluated the difference in the fragmentation rate as a function of whether the nucleic acids to be fragmented are double strand RNA or DNA or a RNA/DNA heteroduplex (FIG. 7). In this case, for the three controls and as seen in FIG. 6, we observe an increase in fluorescence from the formation of the duplexes then during the fragmentation induced by adding ascorbate, a reduction in fluorescence which meets the level of fluorescence of a completely cleaved single strand.

In all cases and regardless of the nature of the hybrid, fragmentation takes place, showing that the ClipPhen-Cu/ascorbate mixture is entirely suited to the degradation of any type of nucleic acid.

### EXAMPLE 5

### Decontaminating Effect of a Fragmentation Molecule Immobilized on a Solid Support

### Aim:

To immobilize ClipPhen on a solid support and demonstrate that its activity is maintained after immobilization on Tentagel polystyrene beads (trade reference MB 250 002, Rapp Polymers, Germany).

### Operating Procedure:

61.5 mg of Tentagel NH₂ macrobeads (trade reference MB 250 002, Rapp Polymers, Germany) were placed in a Snap Fit column (ABI, USA) or a Handee Centrifuge column (reference 69705, Pierce, USA), namely n=16.6 μmol. The beads were washed with anhydrous dimethylsulfoxide (DMSO) in order to remove any possible trace of water. The DMSO was extracted. It was placed in a stream of argon. 14 μL of triethylamine (6 eq, 99 μmol, ≅150 mM) in 600 μL of anhydrous DMSO (1 min) was percolated through. This step ensured that the amine groups were properly in the NH₂ form and not in the NH₃⁺ form. The solution was extracted with triethylamine. 36.4 mg of disuccinimidyl suberate (DSS) (6 equivalent, 99 μmol, ≅250 mM, Pierce) in 400 μl of anhydrous DMSO (10 min) was percolated through. The solution containing the excess DSS was extracted.

A ninhydrin test was carried out on a few beads removed at this stage of the reaction in order to ensure that all of the amine functions had reacted with the linker. If this was not the case, the beads would turn blue.

A reaction mixture containing the following was percolated through for a minimum of 1 h (1 h 15-1 h 30) at ambient temperature: 3-ClipPhen (2.8 eq, 45 μmol, 56 mM), 4-dimethylaminopyridine (2.8 eq, 45 μmol, 56 mM), 6.3 μL of triethylamine (2.8 eq, 45 μmol, 56 mM) in 800 μL of DMSO.

It was possible to remove a few μL of reaction mixture before and after reaction in order to determine the degree of functionalization of the beads by differential assay under UV at 328 nm. The reaction mixture had a brown coloration due to the 3-ClipPhen. Washing with anhydrous DMSO was carried out until this brown coloration of the washing solution disappeared.

The initially yellowish beads had a yellow color which was slightly darker after reaction. 5.9 μL of ethanolamine (10 eq, 99 μmol, ≅130 mM) in 800 μL of anhydrous DMSO was added in order to inactivate all of the NHS groups which had not reacted. It was allowed to act for 10 minutes. The beads were washed 5 times with 1 ml of DMSO, and extracted with DMSO. The same operation was repeated with acetonitrile (in order to be able to dry the beads). It was flushed with argon then evaporated with a rotary evaporator.

Complexing with Copper

1 mL of a solution of 1M CuCl₂ in 50/50 DMSO/water was percolated through (on 55 mg of beads, i.e. 145 equivalents with respect to the degree of functionalization) for 10 minutes. It was washed with DMSO and water until the washing solution was colorless (if the initial CuCl₂ solution had been colored), then with acetonitrile. It was flushed with argon. It was dried with a rotary evaporator.

### Cleavage Test

The cleavage test was carried out as follows: the desired quantity of resin, between 1 mg and a single bead, i.e. a quantity of ClipPhen introduced of 125 nmol/12.5 mM for the Tentagel (i.e. a quantity of ClipPhen introduced of 1.25 to 2.5 nmol/125 μM to 250 μM) was introduced into an Eppendorf tube (maximum content 200 μL). The target RNA (1600 base 16s RNA transcripts, concentration between 6 and 70 ng/μL depending on the experiments) was added to the resin, similarly for the ascorbate at the desired concentration (5 mM). The samples had a total volume of 10 μL; the buffer used was a tris buffer with a pH of 7.4, with a final concentration of 20 mM.

A single bead of Tentagel resin sufficed to observe cleavage. “Target alone” (electrophoregram A FIG. 8), “target in the presence of ascorbate” (electrophoregram B), “target in the presence of resin” coupled with ClipPhen-Cu complex control samples were produced. The “target in the presence of ascorbate” control, meant that the fact that the quantity of ascorbate used has no effect on fluorescence could be checked. The prepared samples were agitated for 30 minutes at ambient temperature (1000 rpm) before being deposited on an Agilent RNA 6000 Nano chip (commercial kit with trade reference 5067-1512, Agilent, USA), depending on the target used. 1 μL of supernatant was sufficient for analysis on a Bioanalyzer (Agilent, USA).

### Results and Conclusions:

The results obtained and shown in FIG. 8 (electrophoregram C) show that cleavage of the target did indeed occur within half an hour, only in the presence of ascorbate, for a minimal quantity of resins (just 1 bead of Tentagel, i.e., assuming that 1 mg of resin contains 50 to 100 beads, a quantity of supported ClipPhen in the range 1.25 to 2.5 nmol, i.e. a concentration in the range 125 to 250 μM). This cleavage was very readily observable on the RNA model since the fragments which are generated are visible on the electrophoregram (electrophoregram C).

Thus, it has been demonstrated that the immobilized ClipPhen is still active and retains its properties of nucleic acid degradation.

### EXAMPLE 6

### Demonstration of Decontamination of a Solution Containing Contaminating Nucleic Acids and Nucleic Acids of Interest

### Aim:

To eliminate “genomic cellular DNA” contaminants found in the supernatant from a viral culture without degrading the viral nucleic acids present in smaller quantities and protected by a viral capsid, with the aim of reducing non-specific amplifications which occur from cellular DNA without inhibiting the specific amplification of the target of interest.

To this end, before and after action of the ClipPhen-Cu/ascorbate mixture, the concentration of genomic 18S DNA in the supernatant was assayed (Search LC+genomic DNA commercial assay kit, Promega, reference G3041, USA) to produce a range of dilutions. At the same time, the concentration of viral RNA before and after the action of the ClipPhen-Cu/ascorbate mixture was assayed by PCR using specific primers and assayed with Light Cycler, SybrGreen (Roche, USA).

### Operating Procedure:

Cells infected with a virus were cultured. In a control experiment, the residual genomic DNA (18S) in the supernatant was assayed using the Kit Search LC (entry B in the table below). The quantity of nucleic acids corresponding to the virus was also measured using the Light Cycler (Roche) (amplification technique using specific primers) (entry B in the table below).

In another experiment (entry A in the table below), the supernatant was subjected to the action of the ClipPhen-Cu/ascorbate mixture (ClipPhen-Cu 100 μM, ascorbate 50 mM, 30 min, DTT inactivation with 2.5 M final) for 30 minutes, then the quantity of residual genomic DNA (18S) and the quantity of nucleic acids corresponding to the virus were measured.

| | | | | Viral RNA | |
|---|---|---|---|---|---|
| | Genomic 18S DNA quantification | | | quantification | |
| | (LC Search test) | | | (Light Cycler) | |
| | | Cycle no | pg/μL | cp/cap | Entry |
| ClipPhen- | Supernatant + | 19 | 2600 | 2500 | A |
| Cu/ | ClipPhen | | | | |
| ascorbate | Supernatant − | 14.8 | >26000 | 3000 | B |
| | ClipPhen | | | | |

### Results and Conclusions:

It was observed that in the case in which the supernatant was treated with the cleaving agent, the concentration of genomic DNA was 10 times lower (entry A) without the concentration of viral nucleic acid being substantially affected.

This technique can thus increase the sensitivity of assaying viral nucleic acids, by substantially reducing the background noise.

1. A method for decontaminating a solution of any nucleic acid present in said solution, comprising the following steps:
for a sufficient period, subjecting the solution to the action of at least one fragmentation molecule formed by a complex constituted by two molecules of 1,10-phenanthroline associated with a metal atom forming the bis(1,10-phenanthroline)/metal complex until said nucleic acids have been completely degraded; and
stopping the activity of the fragmentation molecule by adding:
an excess of organic reducing agent; or
a complexing agent such as EDTA.

2. A method for decontaminating a solution containing contaminating nucleic acids and nucleic acids of interest, comprising the following steps:
for a sufficient period, subjecting the solution to the action of at least one fragmentation molecule formed by a complex constituted by two molecules of 1,10-phenanthroline associated with a metal atom forming the bis(1,10-phenanthroline)/metal complex until said contaminating nucleic acids have been degraded, while conserving the nucleic acids of interest; and
stopping the activity of the fragmentation molecule by adding:
an excess of organic reducing agent; or
a complexing agent such as EDTA.

3. The method as claimed in claim 1, wherein after stopping the activity of the fragmentation molecule, the method comprises a supplemental step which consists in an amplification reaction.

4. The method as claimed in claim 1, wherein the metal is a transition metal such as copper, ruthenium, nickel, iron, zinc, rhodium, cobalt or manganese.

5. The method as claimed in claim 1, wherein the two phenanthroline nuclei of the fragmentation molecule are connected to each other via a linking arm to form a ClipPhen molecule.

6. The method as claimed in claim 5, wherein the linking arm between the two phenanthroline nuclei is constituted by a chain of three successive carbon atoms wherein the carbon atom in the central position is substituted and wherein each terminal carbon atom is connected to a phenanthroline nucleus via an oxygen atom.

7. The method as claimed in claim 6, wherein the carbon atom in the central position is substituted with —NH₂ or —NH—CO—CH₃ and in that each terminal carbon atom is connected to a phenanthroline nucleus via an oxygen atom in position 2 or 3 of said nucleus.

8. The method as claimed in claim 5, wherein the ClipPhen molecule is a 3-ClipPhen (1,3-bis(1,10-phenanthrolin-3-yloxy)propan-2-amine).

9. A method for decontaminating a solution of any nucleic acid present in said solution, consisting of subjecting the solution to the action of the ClipPhen/metal molecule for a sufficient period until said nucleic acids, termed contaminating nucleic acids, have been completely degraded.

10. A method for decontaminating a solution containing contaminating nucleic acids and nucleic acids of interest, consisting of subjecting the solution to the action of the ClipPhen/metal molecule for a sufficient period until said contaminating nucleic acids have been degraded, while conserving the nucleic acids of interest.

11. The method as claimed in claim 1, wherein the fragmentation molecule is a type I copper complex associated with hydrogen peroxide, H₂O₂, and with another reducing agent.

12. The method as claimed in claim 1, wherein the molecule is a type II copper complex associated with another organic reducing agent.

13. The method as claimed in claim 1, wherein the reducing agent is an organic reducing agent constituted by:
a thiol such as dithiothreitol (DTT), a thioacid such as mercaptopropionic acid; or
a carboxylic acid; or
a derivative of a carboxylic acid such as the ascorbate; or
a phosphine such as tricarboxyethyl phosphine (TCEP); or
a combination of at least two of said organic reducing agents.

14. The method as claimed in claim 1, wherein the fragmentation molecule is immobilized on a solid support.

15. The method as claimed in claim 1, wherein the reducing agent S or the other reducing agent is immobilized on a solid support.

16. The method as claimed in claim 14, wherein the solid support is a particle, a membrane, a strip, a film or a filter.

17. The method as claimed in claim 11, wherein the ratio between the fragmentation molecule and the other organic reducing agent, constituted by a carboxylic acid or a derivative of said acid, is in the range between 1 to 1 and 1 to 100.

18. The method as claimed in claim 1, in which the ratio between said fragmentation molecule and the reducing agent constituted by a thiol, is more than 1 to 100.

19. The method as claimed in claim 7, wherein the solution contains all of the constituents necessary for an amplification reaction with the exception of the nucleic acids, i.e.:
targets;
amplification primers; and
detection probes.

20. The method as claimed in claim 1, wherein the nucleic acids which are treated are RNA or DNA in the single or double strand form, as well as RNA/DNA heteroduplexes.

21. The method as claimed in claim 1, wherein the period for the treatment undergone by the treated nucleic acids is in the range 5 to 60 minutes for concentrations of ClipPhen/metal in the range 1 μM to 100 μM.

22. The method as claimed in claim 1, wherein the solution is mixed with a biological sample containing target nucleic acids which are to be amplified, and amplification primers and detection probes which are specific for the target nucleic acids in the presence of the organic reducing agent thereby creating an excess of organic reducing agent which stops the action of the fragmentation molecules.

23. The method as claimed in claim 22, wherein the organic reducing agent as is identical to the organic reducing agent for stopping the activity of the fragmentation molecule.

24. The method as claimed in claim 22, wherein the organic reducing agent is different from the organic reducing agent for stopping the activity of the fragmentation molecule.
